(19) United States
(12) Plant Patent Application Publication
(10) Pub. No.: US 2016/0128255 P1
(43) Pub. Date: May 5, 2016

(54) Pomegranate tree named 'RED JAY'

(50) ;

(71) Applicant: Y & Y MANAGEMENT COMPANY, LLC, Bakersfield, CA (US)

(72) Inventor: Jeffrey A. Yurosek, Bakersfield, CA (US)

(21) Appl. No.: 14/121,924

(22) Filed: Nov. 4, 2014

(51) Int. Cl. A01H 5/00

(52) U.S. Cl. PLT/210

(57) ABSTRACT

A new and distinct pomegranate variety named ‘Red Jay’, characterized by having a very early fruiting trait and dark red coloration in both internal seed arils and external skin, which is characterized as being mostly similar to a ‘Wonderful’ (unpatented) variety except that it is ready for harvest approximately 60 days prior and has darker red skin and seed aril coloration.

### CLASSIFICATION

The present invention relates to a new and distinct variety of pomegranate known botanically as Punica granatum.

### VARIETY DENOMINATION

The new plant has the varietal denomination ‘RED JAY’.

### BACKGROUND OF THE NEW VARIETY

The present invention relates to a new and distinct variety of pomegranate tree referred to hereafter as the ‘Red Jay’ pomegranate tree, broadly characterized by its very early maturing habit, low acid content when ripe, and the deep red color of both the skin and seed arils.

The new variety of the present invention was discovered as a sport growing on a Wonderful (unpatented) pomegranate tree in a commercial planting in the inventor's farm near McFarland, Calif., USA. The instant variety was then asexually reproduced by cuttings made from the original sport with the resulting trees and fruit being identical to that original sport.

The fruit of the instant variety are mature for commercial harvest and shipment approximately August 5-15 or 15-25 days prior to the pomegranate variety ‘Smith’ (U.S. Plant Pat. No. 16,578) and 60 days prior to pomegranate variety ‘Wonderful’ (unpatented) in McFarland, Calif., USA. The new fruit is 10-15 days earlier maturing and has a distinctively darker skin and aril color when ripe than the ‘Early Foothill’ (unpatented). The new fruit is also 5-10 days earlier maturing with a darker red external and internal seed aril color than ‘Granada’ (U.S. Plant Pat No 2,618).

The State of California marketing regulations requires that pomegranate fruit may not be marketed until the juice from the arils have an acid content of no more than 1.85%. This new variety is able to meet this acid level requirement by July 25, however the fruit does not reach a marketable color or brix level until early August. While there are not any regulations related to required color, the market and consumers will generally not accept fruit and more specifically internal seed arils, which are not red in color. By August 5, the instant variety is dark red externally with acceptably red internal seed arils. Early, sweet, and dark red colored fruit is desired by consumers and marketers and will generally attract higher prices for the grower, which makes this novel trait extremely desirable.

### SUMMARY OF THE INVENTION

The new variety of pomegranate is generally characterized as having similar tree characteristics to the variety ‘Wonderful’ (unpatented), of which it is a sport, with exceptions that the instant variety matures 60 days earlier, has much darker seed aril and fruit skin color when mature and has a shorter petiole length. Skin and seed aril color is darker red than varieties ‘Smith’ (U.S. Plant Pat. No. 16,578), ‘Granada’ (U.S. Plant Pat. No. 2,618), and ‘Early Foothill’ (unpatented). Skin and seed aril color are more uniform throughout the fruit than variety ‘Wonderful’ (unpatented), which can have pink to white shoulders with varying seed aril color and variety ‘Smith’ (U.S. Plant Pat. No. 16,578), which tends to have pink to bright red shoulders and pink to red seed arils even when testing at acceptable acid levels. The new variety tree has medium to high vigor for pomegranate trees of similar age with an upright fountain shape and is slightly more vigorous than the variety ‘Wonderful’ (unpatented) when at the same maturity. Trunk surface is smooth similar to ‘Wonderful’ (unpatented) and more smooth than variety ‘Smith’ (U.S. Plant Pat. No. 16,578). Sugar content in early August will commonly test around 16-18 degrees brix, whereas ‘Smith’ (U.S. Plant Pat. No. 16,578) variety will have a brix level of 10-12 and ‘Wonderful’ (unpatented) variety a brix level of 5-7 at the same time. Inflorescence period of ‘Red Jay’ starts in mid April and will usually reach 80% bloom by late April or early May.

TABLE 1

| | RED JAY | WONDERFUL | SMITH |
|---|---|---|---|
| Bloom | Late Apr-Early May | Early-Mid May | Early May |
| Fruit Maturity | Early-Mid Aug | Early Oct | Early-Mid Sep |
| Skin Color | Dark Red | Red | Bright Red/Pink |
| Aril/Seed Color | Very Dark Red | Red | Bright Red |
| Seed Hardness | Medium | Medium | Soft |

### BRIEF DESCRIPTION OF PHOTOGRAPHS

FIG. 1 is a side view of a typical fruit from ‘Red Jay’;

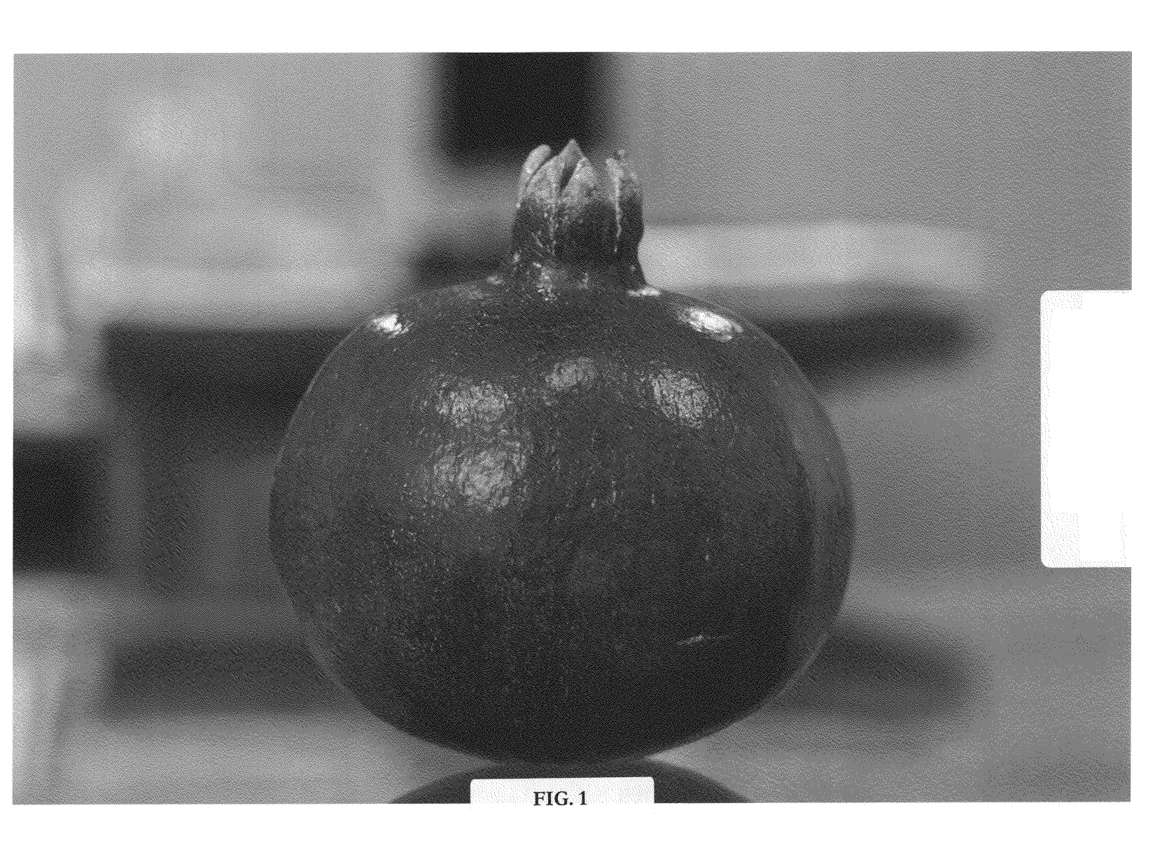

FIG. 2 is a section of a typical piece of fruit from ‘Red Jay’ with seed arils exposed;

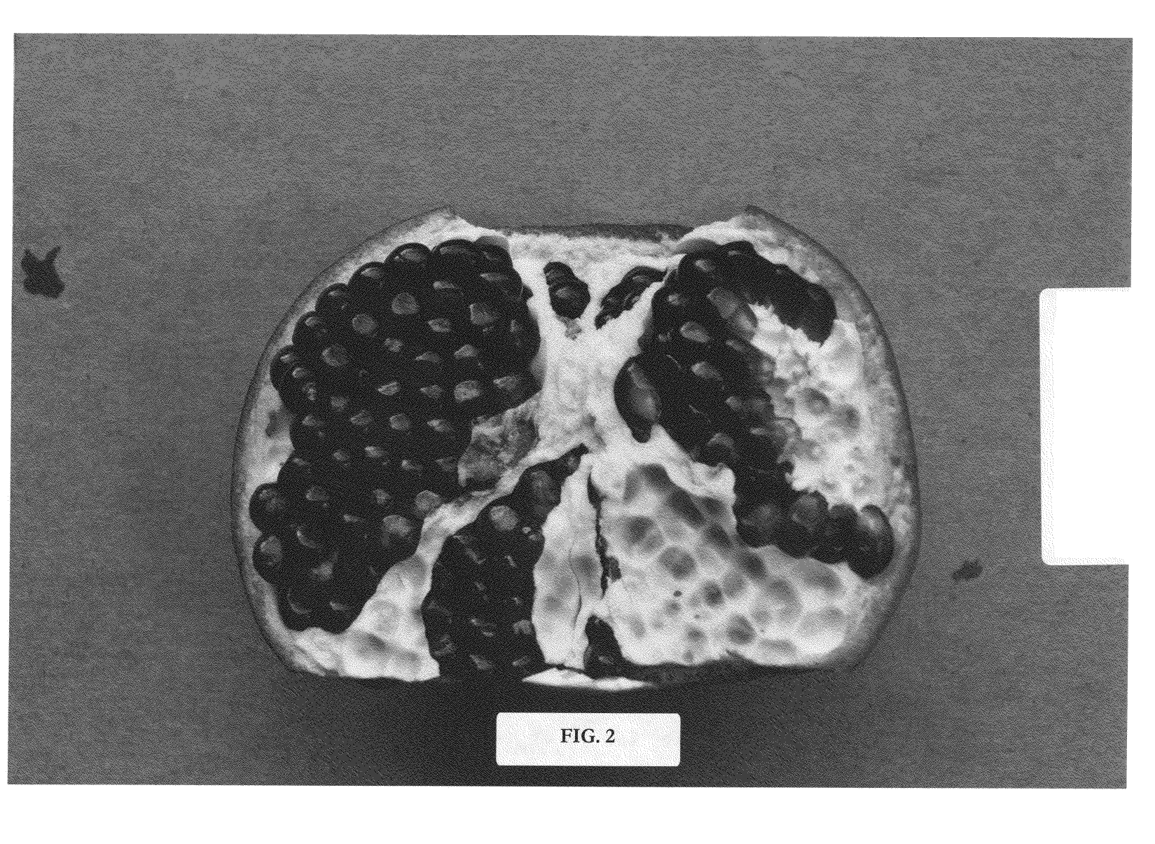

FIG. 3 is a side view of a typical 3-year-old tree of ‘Red Jay’;

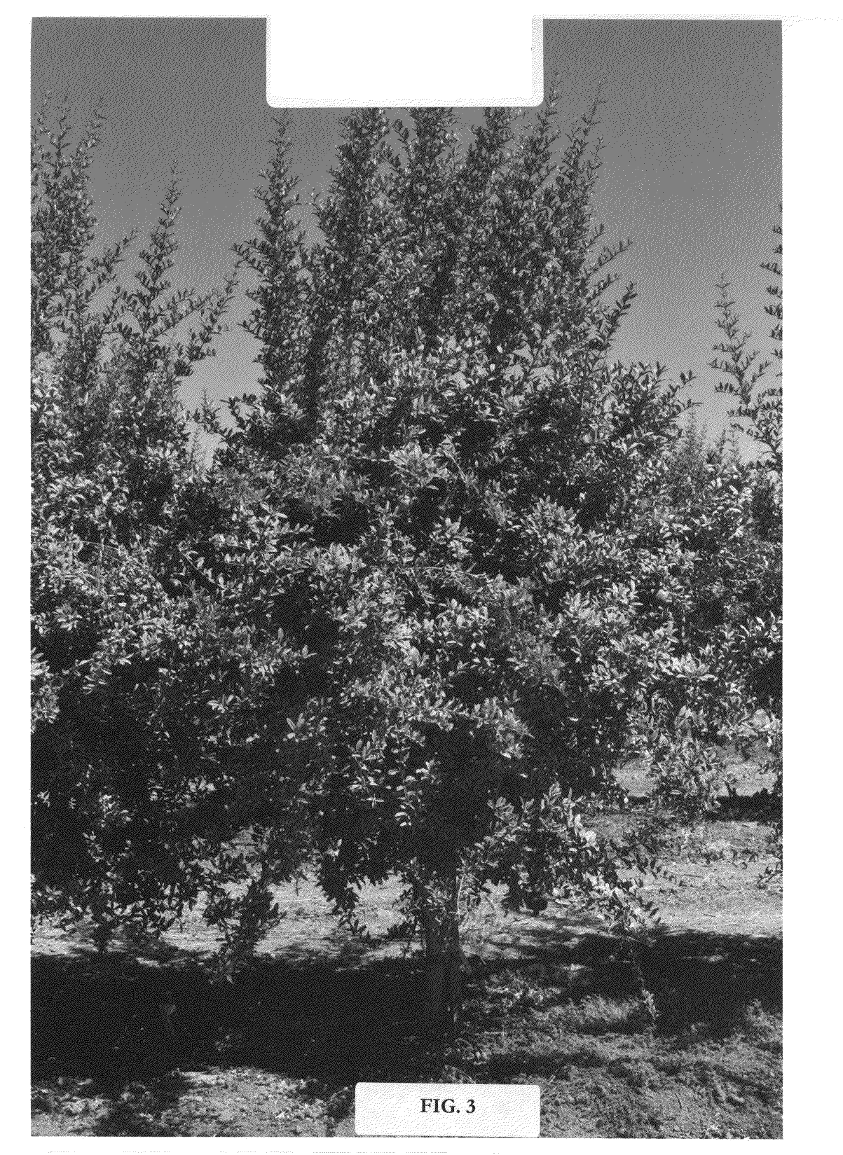

FIG. 4 is a close up view of a typical trunk of a three-year-old ‘Red Jay’;

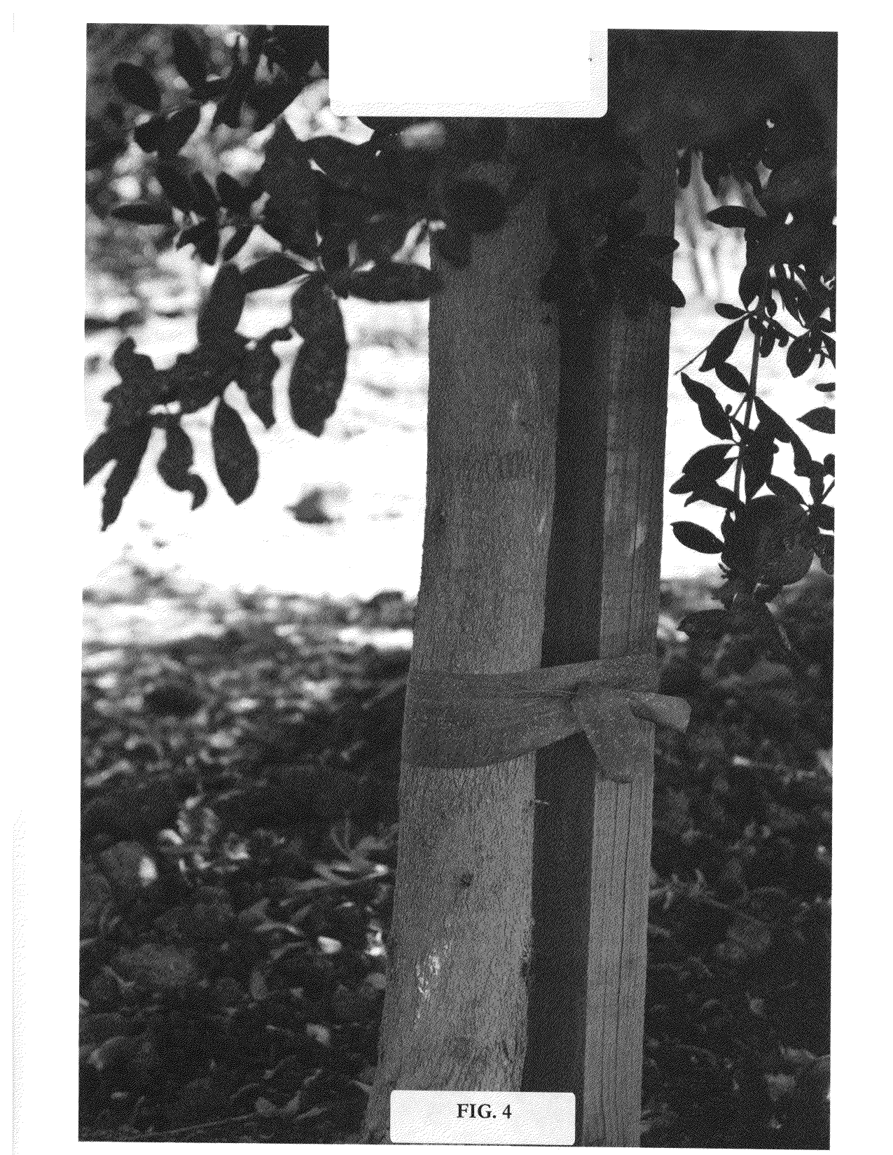

FIG. 5 is a close up view of fruit hanging in a ‘Red Jay’ tree; and

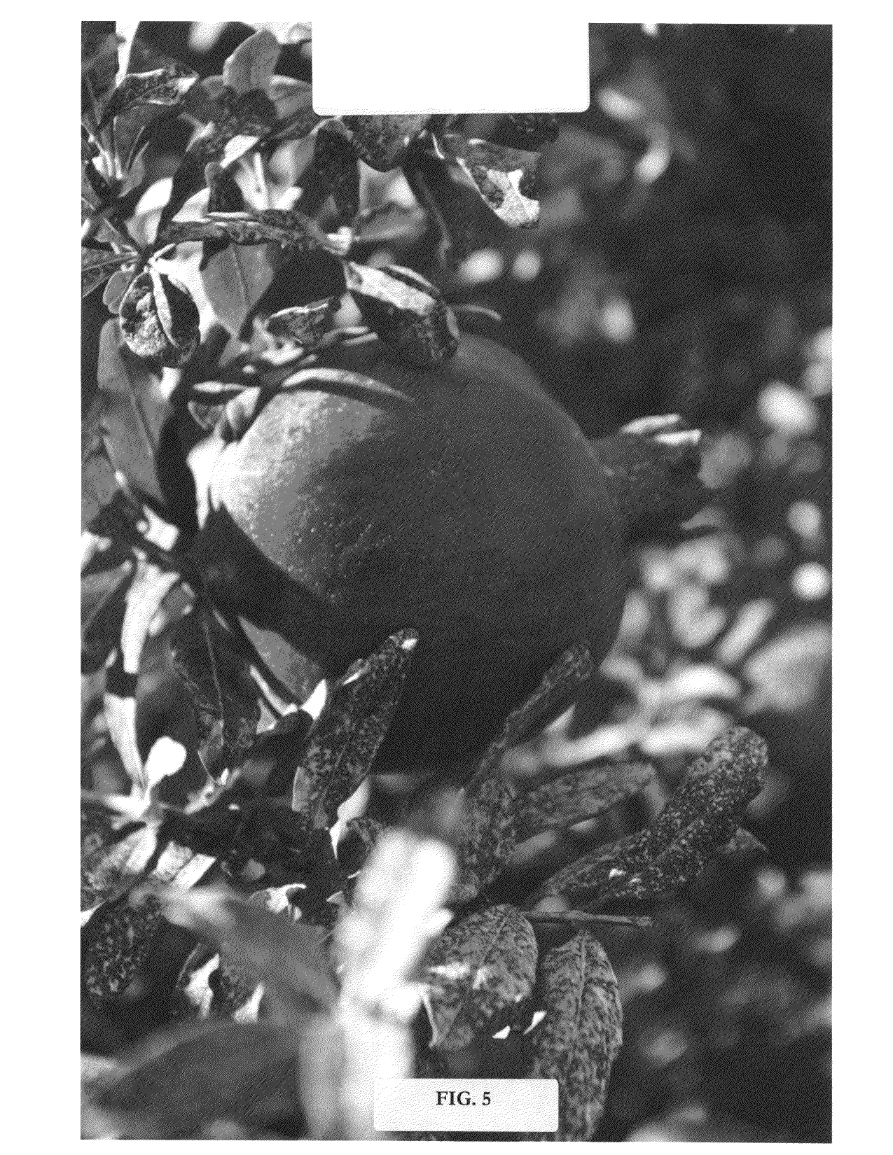

FIG. 6 is a close up side view of a typical flower of ‘Red Jay’.

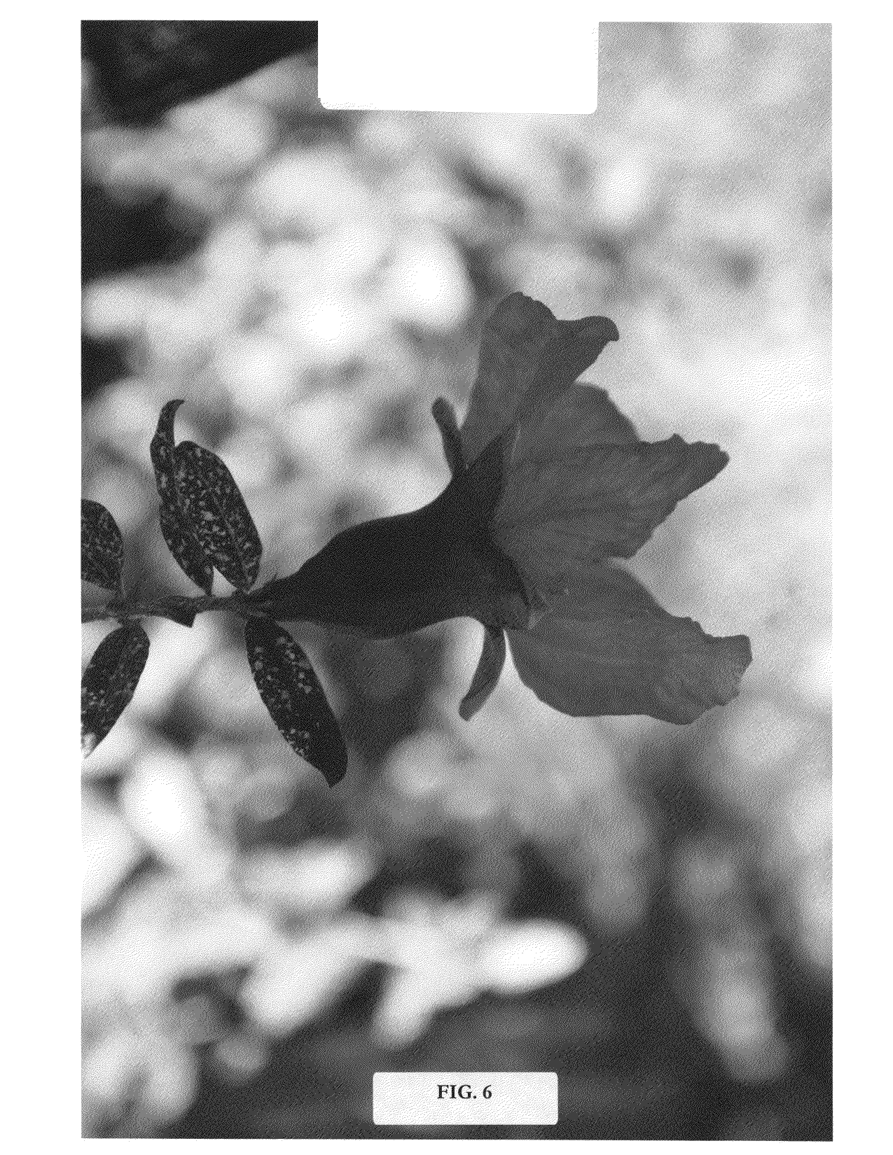

### DETAILED BOTANICAL DESCRIPTION

The following has been observed about the new variety ‘Red Jay’ during the last 3 years in the area of McFarland in Kern County in the Southern San Joaquin Valley of California. Phenotypical expressions may vary depending on changing environmental and climatic factors such as temperature, rainfall, and day length or by introduced external factors such as pruning, training, fertilization, and irrigation. All observations are from 3 year old trees unless otherwise stated.
Tree: Deciduous and not alternate baring
   Size.—normal for pomegranate 10-12 feet.
   Vigor.—medium to high — slightly more vigorous than ‘Wonderful’.
   Figure.—Upright fountain shape similar to ‘Wonderful’.
   Trunks.—Smooth slightly cracked surface appearance with 1 main trunk that easily produces suckers similar to ‘Wonderful’ but more smooth than pomegranate variety ‘Smith’.
   Trunk diameter at 30 cm.—60-65 mm similar to ‘Wonderful’.
   Color.—RHS 199 D.
   Branch diameter (where bloom begins).—6.00-6.35 mm.
   Branches.—slightly thorny.
   Color.—RHS 199 B.
   New growth.—four angled.
Foliage:
   Leaf growth habit.—Symmetrically opposing, simple leaf.
   Petiole.—1 mm in length — similar to ‘Wonderful’ but longer than varieties ‘Granada’ and ‘Smith’. Color: RHS 150 A.
   Leaf shape.—Slightly downward cupping with prominent midrib.
   Texture.—Smooth glossy upper, matte under.
   Leaf color.—RHS 143 A Upper, 143 B Under.
   Leaf origin.—Thorn.
   Leaf length.—38-40 mm.
   Leaf width.—9.5-10 mm.
   Venation. type—Pinnate Color: RHS 142 B.
   Stipule.—RHS 150 B.
   Glands.—None.
Flowers:
   Full bloom length.—52 mm (Beginning of sepal to flower tip).
   Open bloom width.—47 mm.
   Blooming period.—Early-Mid April or 10-15 days before ‘Smith’ variety and 20+ days before ‘Wonderful’ variety.
   Bloom habit.—2-5 per cluster, some singular, closed compact bloom, tubular.
   Calyx depth.—30-35 mm.
   Calyx width.—38-40 mm.
   Calyx base color.—RHS 53 A.
   Sepal length.—13.5 mm.
   Sepal color.—RHS 48 B 5-7 Sepals on average.
   Petal length.—13-14 mm.
   Petal width.—19-20 mm.
   Petal shape.—Oval.
   Petal color.—RHS 32 A.
   Stamen length.—6-8 mm.
   Stigma length.—10-13 mm.
   Ovary length.—16 mm.
   Ovary width.—12-13 mm.
   Male and hermaphrodite flowers.—self and cross pollinating.
Fruit:
   Maturity.—Aug 5-10 or 20-30 days prior to ‘Smith’ variety and 60+ days prior to ‘Wonderful’ variety.
   Size.—82.5 to 90 mm in diameter and slightly smaller than variety ‘Wonderful’.
   Axel length.—60 to 80 mm.
   Aril width.—4-5 mm.
   Aril length.—8-9 mm.
   Shape.—Normal for pomegranates, round shape with protruding calyx.
   Average.—450-500 g.
   Skin color.—46-A RHS.
   Aril color.—53-A RHS.
   Seed color.—NN-155 B RHS.
Market: Fresh market for both whole fruit consumption and the processing of the aril seeds, and for juicing and supplement manufacturing.

I claim:

1. A new and distinct pomegranate tree variety as illustrated herein and described and characterized by having fruit which is substantially earlier maturing and darker red both in external and internal seed aril color.
